(19) United States
(12) Patent Application Publication
(10) Pub. No.: US 2012/0134924 A1
(43) Pub. Date: May 31, 2012

(54) Micro-Vesicles Providing Contrast To Target Tissue Electrical Property Gradients

(75) Inventors: Stephen Anthony Cerwin, Mico, TX (US); Jane F. Emerson, Irvine, CA (US); David B. Chang, Tustin, CA (US); Martin Fessenmaier, Aliso Viejo, CA (US); Nicholas J. Witchey, Laguna Hills, CA (US)

(73) Assignee: MAGNETUS, LLC, Irvine, CA (US)

(21) Appl. No.: 12/956,519

(22) Filed: Nov. 30, 2010

(51) Int. Cl. A61K 49/00

(52) U.S. Cl. 424/9.1

(57) ABSTRACT

Micro-vesicles that become acoustically sensitive in the presence of a Radio Frequency (RF) Electromagnetic (EM) field are presented. The micro-vesicles can comprise a main body having one or more affinity ligands configured to preferentially bind to a target tissue. Once bound, the micro-vesicles and the target tissue can be bathed in an RF EM field, which induces the target tissue or micro-vesicles to generate an acoustic signal. The micro-vesicles can also become receptive to acoustic energy. An acoustic therapeutic signal can be directed toward the target tissue and micro-vesicles, which causes therapeutic excitation of the micro-vesicles. The therapeutic excitation can include heating the target tissue, releasing a drug formulation, or other excitation. The disclosed techniques can be used with a high degree of precision to activate micro-vesicles local to the target tissue.

### FIELD OF THE INVENTION

The field of the invention is acoustic-based therapeutic treatment technologies.

### BACKGROUND

When a patient suffers from internal issues, cancer for example, a health care provider in most instances prefers to treat one or more of the affected internal tissues in a non-invasive manner. In many cases a patient requires major surgery to treat a target tissue (e.g., a tumor). If the patient is treated with chemotherapy systemically rather than locally, the overall adverse side effects are more likely. A preferred approach would include treating just a specific target tissue within the patient without affecting other non-target tissues and without surgery.

One approach that others have taken to target cancer cells includes manufacturing minicells that can be used to carry one or more drug formulations used in chemotherapy. For example, the paper titled “Bacterially Derived 400 nm Particles for Encapsulation and Cancer Cell Targeting Chemotherapeutics” by MacDiarmid et al. (Cancer Cell 11, 431-445, May 2007), describes that minicells attach to a targeted cancer cell to deliver bioactive formulations. Unfortunately, the minicells are passively activated and thus are not be under complete control of a practitioner. It is desirable to include a controlled activation step such that a micro-vesicle is activated only when properly positioned at a target site.

One little known area of tissue characterization includes identifying target tissues by their conductivity. For example, international patent application publication WO 2005/057467 to Jersey-Willuhn et al. titled “Tissue Characterization Using an Eddy Current Probe”, filed Dec. 1, 2004, describes techniques for mapping electrical conductivity of biological tissues. Conductivity gradients within tissues, or between tissues, provide a handle for targeting a specific tissue through Electro-Magnetic Acoustic Imaging (EMAI) where the tissue is bathed in a Radio Frequency (RF) electro-magnetic field. The RF field induces gradients to generate internally sourced ultrasounds, which can then be used for imaging the target tissue. One should note the resulting ultrasound images reflect conductive properties of the tissue in addition to tissue density as in conventional ultrasounds.

The applicants have pioneered the use of EMAI as discussed in co-owned U.S. Pat. No. 6,535,625 to Chang titled “Magneto-Acoustic Imaging” filed Sep. 24, 1999; U.S. Pat. No. 6,974,415 to Cerwin titled “Electromagnetic Acoustic Imaging” filed May 22, 2003; U.S. Pat. No. 7,122,009 to Chang titled “Blood Pressure Diagnostic Aid” filed Sep. 3, 2004; U.S. patent application publication 2007/0038060 titled “Identifying and Treating Bodily Tissues Using Electromagnetically Induced, Time-Reversed, Acoustic Signals” filed Jun. 9, 2006; and U.S. patent application having Ser. No. 12/786,232 filed May 10, 2010.

As one could imagine in view of the infancy of EMAI technology, no effort has been put forth toward combining EMAI techniques with drug delivery via micro-vesicles. Previously, some work showed that nanomagnetic particles can be manufactured for use with ultrasound therapies or magnetic fields. For example, U.S. patent application publication 2004/0210289 to Wang titled “Novel Nanomagnetic Particles”, filed Mar. 24, 2004, describes nanoparticles configured for use to deliver drug formulations. Although Wang discusses using electromagnetic energy and acoustic energy, Wang fails to appreciate that both types of energy can be used substantially at the same time and location to activate micro-vesicles where the micro-vesicles become acoustically responsive in electromagnetic fields.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

What has yet to be appreciated is micro-vesicles can be formed to bind to a target tissue while enhancing a treatment site's conductivity contrast, or other electrical property, with respect to EMAI applications. Ultrasound induced at a target tissue site from electromagnetic fields can be analyzed to obtain parameters to so generate an acoustic therapeutic signal back toward the micro-vesicles with a high degree of precision at sub-centimeter resolutions. The acoustic therapeutic signal can cause therapeutic excitation of the micro-vesicles, which in turn treats the target tissue.

Thus, there is still a need for micro-vesicles that are acoustically responsive to electromagnetic fields.

### SUMMARY OF THE INVENTION

The inventive subject matter provides apparatus, systems and methods in which one can provide a therapeutic treatment to a target tissue through using micro-vesicles having configured electrical properties sufficient to allow generation of an acoustic signal in the presence of an external RF EM field. One aspect of the inventive subject matter includes a micro-vesicle comprising a main body comprising one or more materials and affinity ligands coupled with the core material. The affinity ligands can be configured to have an affinity to a target tissue over no target tissues. The core material can be configured with electrical properties (e.g., permittivity, permeability, conductivity, etc.) of sufficient value to generate acoustic signals in the presence of an external RF EM field. Preferably, the generated acoustic signals from the micro-vesicles are at least a two-fold increase in strength relative to an acoustic signal induced in the target tissue from the external RF EM field, thus increasing the contrast of the tissue.

Contemplated micro-vesicles can also include one or more drug attachment sites. One or more drug formulations can be bound to the micro-vesicles at the binding sites as desired. When the micro-vesicle binds to a target tissue via the affinity ligands, the bound drug formulations are thus in desirable close proximity to a treatment site. In some embodiments the drug attachment sites are configured to release the drugs when the micro-vesicle is exposed to an acoustic therapeutic signal.

The drug attachment sites can be positioned about the micro-vesicle according to a desired treatment. In some embodiments, the drug attachment sites are on an exterior surface of a micro-vesicle's main body, while in other embodiments, the drug attachment site can include an internal portion or even a cavity of the micro-vesicle.

In some embodiments, the micro-vesicle can be configured to have an acoustic response to the RF field by virtue of its relative conductivity to surrounding tissues. In other embodiments, the micro-vesicle can be affected by the RF fields, which cause the micro-vesicle to become susceptible to acoustic energy. For example, the RF field can cause the material of the micro-vesicle's main body to take on relative conductivity qualities, which provides contrast for EMAI imaging. Furthermore, the mere presence of suitably configured micro-vesicles (e.g., a gold nanoparticle) can also provide contrast for EMAI imaging.

Another aspect of the inventive subject matter is considered to include a method of treating a patient using the contemplated micro-vesicles. The method can include administering a micro-vesicle-based agent to the patient where the micro-vesicles in the agent are configured to be activated in an applied RF EM field and configured to bind preferentially to the target tissue. The method can further include irradiating the target tissue with an RF field, which can cause generation of an induced acoustic signal due to gradients of electrical properties in the tissue, or at least in part due to the presence of the micro-vesicles. Another step of the method can include locating a treatment site on the target tissue by using the induced acoustic signal. An acoustic therapeutic signal can be directed back to the target site to acoustically activate the micro-vesicles.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic overview of a micro-vesicle.

FIG. 2 is a schematic of a micro-vesicle preferentially binding to a target tissue.

FIG. 3 is a schematic of treating a target tissue using micro-vesicle within an EMAI environment.

FIG. 4A is a schematic of a micro-vesicle exhibiting vibration as a therapeutic excitation in response to an acoustic therapeutic signal.

FIG. 4B is a schematic of a micro-vesicle triggering release of a bound drug as a therapeutic excitation in response to an acoustic therapeutic signal.

FIG. 4C is a schematic of a micro-vesicle that is ruptured as a therapeutic excitation in response to an acoustic therapeutic signal causing release of a drug.

FIG. 5 is an example method of treating a patient with a micro-vesicle-based agent.

### DETAILED DESCRIPTION

It should be noted that while the following description is drawn to medical electronic devices (e.g., EMAI devices, phonon lasers, etc.) which can include various alternative configurations and may employ various computing devices including servers, interfaces, systems, databases, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclose apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

One should appreciate that the disclosed techniques provide many advantageous technical effects including treating target tissues within a patent in a non-invasive manner. Furthermore, the disclosed micro-vesicles can provide enhanced conductivity contrast, or contrast in other electrical properties; in an EMAI environment and can provide treatment to a target tissue when suitably triggered by an RF EM field and acoustic energy. When a micro-vesicle is in the presence of an RF EM field, the micro-vesicle can generate ultrasounds providing increased contrast resulting from electrical properties relative to surrounding tissues. One should note the micro-vesicle is not necessarily activated when an RF EM field is not present. For example, a micro-vesicle constructed of some insulator materials (e.g., glass, polymers, porous silicon, gels, etc.) can become acoustically sensitive in the presence of an RF EM field because the micro-vesicle's electrical properties relative to surrounding tissues. In addition, contemplated micro-vesicles can be considered pre-activated by the RF EM field so that when an incident acoustic energy of sufficient amplitude or frequency impacts the micro-vesicle, the micro-vesicle triggers a therapeutic excitation; a drug release, a release of heat, or other excitation.

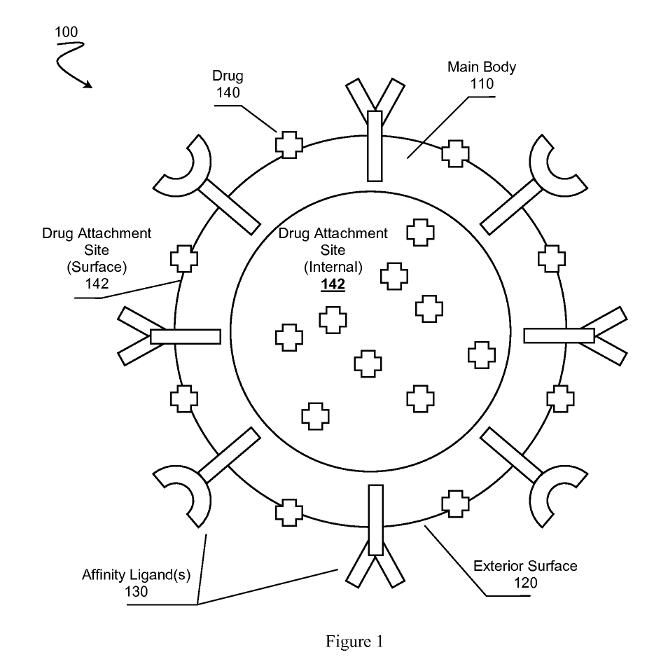

In FIG. 1, micro-vesicle 100 comprises main body 110 having one or more types of affinity ligands 130. Although main body 110 is illustrated as having a cavity, it is also contemplated that main body 110 can be substantially solid. Main body 110 can comprise various materials that preferably become activated in the presence RF EM fields. Example materials can include conductors or non-conductors, where non-conductors are more preferred over conductors. Micro-vesicle 100 is sized and dimensioned to have linear dimension of preferably less than 500 nm, more preferably less than 400 nm, and even more preferably less than 100 nm. One example of particles that could be adapted for use as described below includes the nanomagnetic particles described in U.S. patent application publication 2004/0210289 to Wang titled “Novel Nanomagnetic Particles”. However, main body 110 more preferably comprises at least some non-conductor material, possibly where the non-conductor material becomes conductive in the presence of an EM field as discussed below.

Non-conductor (e.g., insulator) materials can have one or more desirable properties over conductor material. For example, non-conductors can be less toxic over conductors. Furthermore, non-conductor materials can be formulated or otherwise tuned to behave appropriately when exposed to a RF EM field. As discussed below, a non-conductor can exhibit conductor behavior under the presence of an EM field, which can enhance the contrast of a conductivity gradient, or other electrical property gradients. One should note that such behavior is considered more desirable than providing a basic pure conductor-based micro-vesicle 100, which would be affected by EM fields in an undesirable fashion. Although less preferable, a substantially conductor-based micro-vesicle 100 are considered to have advantageous properties as well. For example, a conductor-based micro-vesicle 100 can be used to help destroy a target tissue when in the presence of an RM EM field or acoustic energy.

One should note that an insulator-based micro-vesicle 100 is not required to undergo a transition to a conducting state before micro-vesicle 100 would be effective for generating acoustic signals (e.g., internally induced ultrasound signals). For example, micro-vesicle 100 could be constructed a non-conductor (i.e., a good insulator) and having an electrolyte at the surface, possibly the target tissue or a body fluid. When exposed to RF EM fields, the electrolyte at the surface layer of the non-conductor would generate acoustic signals even as the insulator has not yet transitioned into a conductive state. One should appreciate the inventive subject matter is considered to include a micro-vesicle constructed having a variable propensity for generating acoustic signals as a function of RF EM field properties (e.g., intensity, amplitude, frequency, etc.) based on varying electrical properties of a core material composing micro-vesicle 100 and discontinuities of the properties relative to adjacent materials (e.g., tissues, fluids, etc.).

One acceptable insulator material that can be adapted for use with the disclosed inventive subject matter includes a glass that becomes a conductive in the presence of high electric fields. Example materials are described in the paper by Lee et al. titled “Liquid Glass Electrodes for Nanofluidics” published on May 16, 2010, Nature Nanotechnology 5, 412-416 (see also URL www.physorg.com/news193298040.html).

An additional example of insulator materials includes porous silicon. U.S. patent application publication 2010/0238297 to Hwu et al. titled “Particles and Manufacturing Methods Thereof”, filed Aug. 22, 2009, describes manufacturing small nanoparticles out of porous silicon, among other materials, where the particles can be 10 nm or less. Porous silicon, as with other materials, is biocompatible with tissues and can be processed or eliminated by the body naturally. Typically, porous silicon can also have a relative permittivity from about 2 to 3.9; however, the relative permittivity can be adjusted by appropriate doping. Furthermore, one should note that main body 110 can be soft rather than hard. For example, micro-vesicle 100 can include a solid silicon gel impregnated with drug 140. As additional examples, glasses (e.g., silicon-based materials) can have a relative permittivity greater than 4, where Teflon™ is about 2.1.

Insulator materials composing micro-vesicle 100 can exhibit other properties allowing for tuning of micro-vesicle 100. For example, a non-conductor that undergoes a phase transition to a conducting state at high intensity or at a specific frequency of an RF EM field could be used. Alternatively, a non-conductor with a high relative permittivity (i.e., greater than 1000) and no exhibiting a phase change could itself provide good contrast. One should keep in mind that EMAI devices identify tissue electrical property gradients, conductivity for example, via collected induced acoustic signals. Therefore, one can tune the material of micro-vesicle 100 to provide enhanced contrast to the gradients in other electrical properties beyond conductivity under desirable RF EM field conditions.

The inventive subject is considered to include configuring the electrical properties of micro-vesicle 100 to have desired properties during use and exposure to RF EM fields. For example, a core material of micro-vesicle 100 can have electrical properties, relative permittivity for example, sufficient to generate an acoustic signal from micro-vesicle 100 in response to an external RF EM field where the generated micro-vesicle acoustic signal is at least a two-fold increase in strength relative to an acoustic signal generated by a target tissue in response to the same RF EM field. The reader should appreciate that various types of micro-vesicles 100 can be constructed to have a broad spectrum of relative signal strengths over a target tissue.

The Applicant's previous efforts have focused on EMAI systems responsive mainly to conductivity gradients within body tissues as conductivity gradients tend to dominate at RF frequencies. Binding micro-vesicle 100 to a target tissue opens additional vistas of utility as micro-vesicle 100 can be constructed to exploit a wider range of gradients in electrical properties.

The reader is reminded that an electromagnetic stress tensor contains terms of the form:

[∈+(σ/iω)]E²+(1/μ)B² [1]

where ∈ is the relative permittivity, σ a is the electrical conductivity, ω is the angular RF frequency, μ is the magnetic permeability, E is the electric field, and B is the magnetic field. Therefore, gradients present ∈, σ, and μ can be leveraged to generate internally induced acoustic signals (e.g., ultrasounds). Again, one should appreciate the concept that gradients or discontinuities in ∈, σ, and μ are leveraged to generate internally induced ultrasounds rather than a simple absolute value of each parameter.

For physiological tissues exposed at RF frequencies, ∈ is on the order of O(10⁻²)(σ/ω), which is typically small relative to the conductivity σ. However, when micro-vesicle 100 is suitably configured, other parameters become more prevalent. For example, if micro-vesicle 100 encapsulates a titanate material with ∈=O(10⁴), the ∈ term can have a larger contribution relative to the σ/iω term thus giving rise to a detectable acoustic signal arising from permittivity gradients. Furthermore, if micro-vesicle 100 encapsulates a high permeability material, then a large gradient in μ becomes relevant. Example high permeability material that can be used in micro-vesicle 100 include mu metal (μ=O(20,000 to 100,000)) or supermalloy (μ=O(100,000 to 1,000,000)). Although the magnetic field term depends on 1/μ, one should appreciate that micro-vesicle 100 having a high permeability relative to an adjacent tissue forms a discontinuity that can be detected.

Acoustic signals are generated where there is a change in the electromagnetic stress tensor. Considered an example based on permeability. The relative permeability of a body tissue is close to unity. When micro-vesicle 100 is configured with a high permeability, it will generate additional contrast. The magnetic stress inside micro-vesicle 100 will likely be small relative to that outside. The difference in magnetic stress will be made up by generating acoustic signals (e.g., ultrasounds) to create a compensating acoustic stress.

With respect to permeability, note

E=O(ωBR) [2]

where ω is the angular RF frequency and R is the radius of the coil being used to generate the electric (E) and magnetic (B) RF fields. Thus the (σ/ω)E² term in the stress tensor can be written (σωR²)B² where (σ/ω)E²>>(σωR²)B². Comparison of this term to the (1/μ)B² term in the stress tensor shows that (1/μ)B² will be large compared to (σ/ω)E² when 1>μσωR².

As a numerical example, consider μ=μ₀ where μ₀=4π×10⁻⁷ Henries/m is the permeability of a vacuum, and take ρ=1 S/m (i.e., an approximate upper limit to tissue conductivities), ω=3×10⁷ sec⁻¹, which corresponds roughly to an RF frequency of 5 MHz, and R=0.05 m, a typical size EMAI coil anticipated for body measurements. Assuming the above parameters, the conductivity-electrical field terms of the stress tensor are much smaller by a factor of 0.094 relative to the magnetic field terms. One should appreciate that the above parameters provide an illustration for a single example while other conditions (e.g., frequencies, tissues, materials, etc.) would yield alternative results.

Thus, the (1/μ)B² term in the stress tensor will be larger than the (σ/ω)E² term in most physiological applications of EMAI. However, μ does not vary appreciably in tissue, remaining always close to μ₀. Thus, since ultrasound generation requires spatial variation of the electromagnetic stress tensor, (1/μ)B² cannot be used to generate ultrasound in tissue that does not contain some injected magnetic material. By contrast, a term like (σ/ω)E² can generate ultrasound in virgin tissue, since σ can vary appreciably in the tissue itself. When micro-vesicles having a large discontinuity μ relative to a tissue is introduced to a tissue, the (1/μ)B² term can also contribute to a generated ultrasound.

In more preferred embodiments, the electrical property values are adjusted as desired to yield an enhanced contrast. To further illustrate this consider yet another example that illustrates how materials can contribute to contrast with a target tissue. Consider a scenario where E and B are normal to a boundary between externally introduced materials (e.g., micro-vesicle 100) and a typical body electrolyte or tissue. Therefore, D=∈_{eff} E and B are continuous across the boundary. Here, E and B are the RF electric and again magnetic fields, respectively, and

∈_{eff}=∈+σ/(iω) [3]

again where ∈=∈_{R}∈₀ is the dielectric constant, where ∈_{R} is the relative permittivity and ∈₀ is the permittivity of free space; σ is the electrical conductivity; and ω is the angular RF frequency.

For the following discussion, assume terms are labeled with subscripts referencing the materials impinged by the RF EM fields. Terms having a subscript of “1” apply to a body material (e.g., electrolyte, tissue, etc.) and terms having a subscript of “2” apply to introduce materials, in this case a materials composing micro-vesicle 100. Then, EMAI pressure (P_{EMAI}), generated at a boundary surface between the introduced material and body material can be expressed as:

P_{EMAI}=∈_{eff1}(E₁²/2)[1−(∈_{eff1}/∈_{eff2})]+(B₁²/2μ₁)[1−(μ₁/μ₂)] [4]

That is, expanding the terms:

P_{EMAI}={∈₁+(σ₁/iω)}(E₁²/2)[1−({∈₁+(σ₁/iω)}/{∈₂+(σ₂/iω)})]+(B₁²/2μ₁)[1−(μ₁/μ₂)] [5]

In the above expression μ=μ_{R}μ₀ denotes magnetic permeability of the material where μ_{R} represent the relative permeability and μ₀ represent the permeability of free space.

Another example set of parameters beyond those previously presented above can include the following: an RF frequency of 5 MHz (ω=3×10⁷ sec⁻¹), permittivity for a body material of ∈>>80/(36π×10⁹) MKS, a nominal value of conductivity of σ>>1 S/m, a tissue permeability of μ₀=4π×10⁻⁷ Henry/m, a number of ampere turns in a Helmholtz coil of NI=1000 amp turns, and a coil radio of R=0.05 m. These parameters yield an approximate RF magnetic field magnitude of B=0.025 Weber and an approximate RF electric field magnitude of E=18,800 v/m. Plugging these field values into equation [5] yield a P_{EMAI} of:

P_{EMAI}{0.125−i5.84}[1−({7.7×10⁻¹⁰−i3.3×10⁻⁸}/{8.8×10⁻¹²∈_{R2}−i3.3×10⁻⁸σ₂})]+250[1−(μ₁/μ₂)]. [6]

One should note from equation [6] the various terms for a body material have the following approximate relative values:

β₁(E₁²/2):(σ₁/iω)(E₁²/2):B₁²/2μ₁=0.021:1:42.8 [7]

Equation [7] illustrates that a dielectric term for the body material is only about 0.021 as large as a conductivity term for the body material, while the conductivity term is 0.023 (1/42.8) as large as the magnetic field term. For typical EMAI environments lacking an introduced material like micro-vesicle 100, the magnetic field terms can be ignored when considering generation of ultrasound because any change in the magnetic permeability from an all-tissue boundary would be negligibly small. However, the situation is reversed when suitability configured micro-vesicles are bound to a body tissue.

From the values in equations [6] and [7] it is apparent that foreign materials having electrical property (e.g., ∈₂, σ₂, μ₂, etc.) values different from the body material can be used to enhance the EM stress in a particular target tissue, which further increases the EMAI induced acoustic signals generated by a treatment site. For example, discontinuities in conductivity of body tissues are usually in about a 0.1 S/m range, so any material causing a larger conductivity gradient would be consider useful.

Preferably electrical property values of micro-vesicle 100 are configured, individually or in combination, to yield an increased acoustic strength of at least two fold. The acoustic strength is proportional to P_{EMAI}, consequently equation [5] provides an approximate formula that one can use as a guide to configure micro-vesicle 100 so that it provides a desired increase in acoustic signal strength relative to a target tissue.

Various types of materials can contemplated to yield the desired effects. Materials having a large relative permittivity can include titannates having ∈_{R}=10,000. While other materials having small relative permittivity can also be used. Equation [6] indicates that even small relative permittivity can yield increased EM stress. Example materials having relatively small relative permittivity include ethyl or methyl acetate (∈_{R}=6), ethyl amine (∈_{R}=6.94), pyridine (∈_{R}=12.3), neoprene (∈_{R}=6.7), non-conducting water (∈_{R}=80), or other materials in addition to those referenced previously.

In view of the large magnitude of the magnetic field term, materials having quite modest magnetic permeability can still have a large impact. Example materials can include rubber containing barium ferrite (μ_{R}=1.05), alnico (m_{R}=4−6), cobalt steel (m_{R}=12), or other materials beyond these or those previously referenced.

Nearly any material having conductivity greater than that of a tissue, which is typically on the order of 0.1 to 1 S/m, would yield acceptable results. Example conductor or semiconductor materials include metal conductors (σ=10⁶−4×10⁷ S/m), carbon or graphite (σ=5×10³−10⁵ S/m), germanium (σ=10−10⁶ S/m), gallium arsenide (GaAs; σ=1−10⁶ S/m), or silicon (Si; σ=1−10⁶ S/m).

Main body 110 can include one or more drug attachment sites 142 where one or more drug formulations can be bound as represented by drug 140. Drug delivery sites 142 are preferably arranged about main body 110 in a manner such that drug attachment sites 142 can be triggered or otherwise activated by acoustic energy. Drug delivery sites 142 can include exterior surface 120, an interior portion of main body 110, within the shell of main body 110, or even impregnated within the material composing main body 110 where main body 110 is substantially solid. As acoustic energy impinges micro-vesicle 100, drug attachment sites 142 activate by simply releasing drug 140, ablating exterior surface 120 or other portions of main body 110, or rupturing main body 110 allowing drug 140 bound internally to be released. Drug attachment sites 142 can be tuned to be acoustically responsive based on properties of an acoustic therapeutic signal including amplitude, frequency, or other property.

Micro-vesicle 100 can also include one or more types of affinity ligands 130 that preferentially bind to a target tissue. Although only two types are shown, one should appreciate fewer, or more, types of affinity ligand 130 can be arranged about main body 110. Affinity ligands 130 can include an antibody targeting the target tissue. For example, micro-vesicle 100 can be configured to target prostate tissue where Prostate Specific Antigen (PSA) is present. The affinity ligands can include anti-PSA antibodies to ensure that micro-vesicle 100 would exclusively bind to the target prostate tissue. Affinity ligands 130 can further include a bi-specific antibody.

More generally, it should be recognized that affinity ligand 130 can be any moiety that has a dissociation constant of equal or less than 10⁻⁵ M, more typically equal or less than 10⁻⁶ M, and most typically equal or less than 10⁻⁷ M. Thus, the particular nature of the affinity ligand is not critical to the inventive subject matter so long as the affinity ligand can bind with high affinity to one or more target structures of a target cell. For example, suitable affinity ligands include antibodies and fragments thereof (e.g., scFV, Fab, F(ab′)₂, etc.), which may or may not be recombinant and/or otherwise genetically modified, receptor-specific ligands and ligand analogs (which may exhibit agonistic or antagonistic effect), competitive or allosteric substrates or ligands. Most preferably, contemplated affinity ligands are also specific with respect to the type or condition of a particular cell or tissue. For example, preferred ligands include those that bind targets that are exclusively or predominantly expressed (or are otherwise present) only on a diseased cell, or on a cell or tissue of a particular tissue type (e.g., bind specifically to PSA for prostate tissue only).

Drug attachment sites 142 are preferably configured to release drug 140 under a therapeutic excitation of micro-vesicle 100 where the therapeutic excitation is caused by an acoustic therapeutic signal. In an EMAI environment, an acoustic therapeutic signal would typically have a different frequency than an input frequency from an RF energy source as discussed further below. Typical input RF EM signal frequencies are in the range from 1 MHz to 100 MHz, more preferably in the range 3 MHz to 20 MHz, and yet more preferably about 5 MHz to 15 MHz. Other frequencies are also contemplated including those greater than 100 MHz, or having more than one dominate peak, each peak at different frequencies.

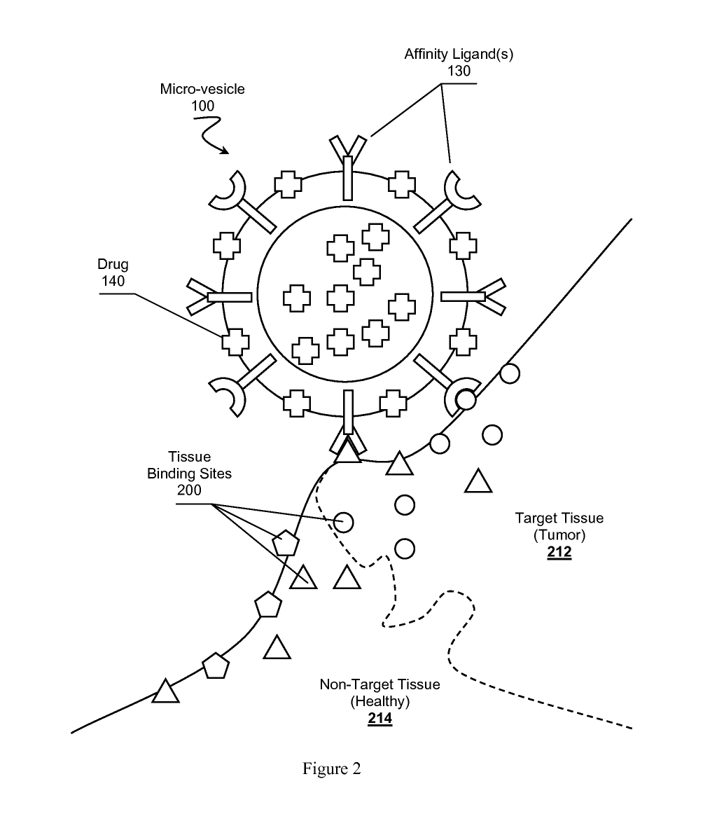

In FIG. 2, micro-vesicle 100 is illustrated after being administered to a patient. In the example illustrated, target tissue 212 represents a tumor while non-target tissue 214 represents neighboring healthy tissue. Both tissues can comprise tissue binding sites 200. However, micro-vesicle 100 has affinity ligands 130 that preferentially bind to target tissue 212. Affinity ligands 130 are configured to have a preference for binding to target tissue 212 over non-target tissue 214 (e.g., healthy tissue) by a preference factor of at least 10, more preferably at least 100, and yet more preferably at least 1000. Furthermore affinity ligand 130 will have a dissociation constant for a target on a target tissue 212 of equal or less than 10⁻⁵ M, and more preferably equal or less than 10⁻⁶ M. Thus, suitable affinity ligands include antibodies and antigen-binding fragments thereof, enzyme substrate analogs, receptor ligands or analogs thereof, etc.

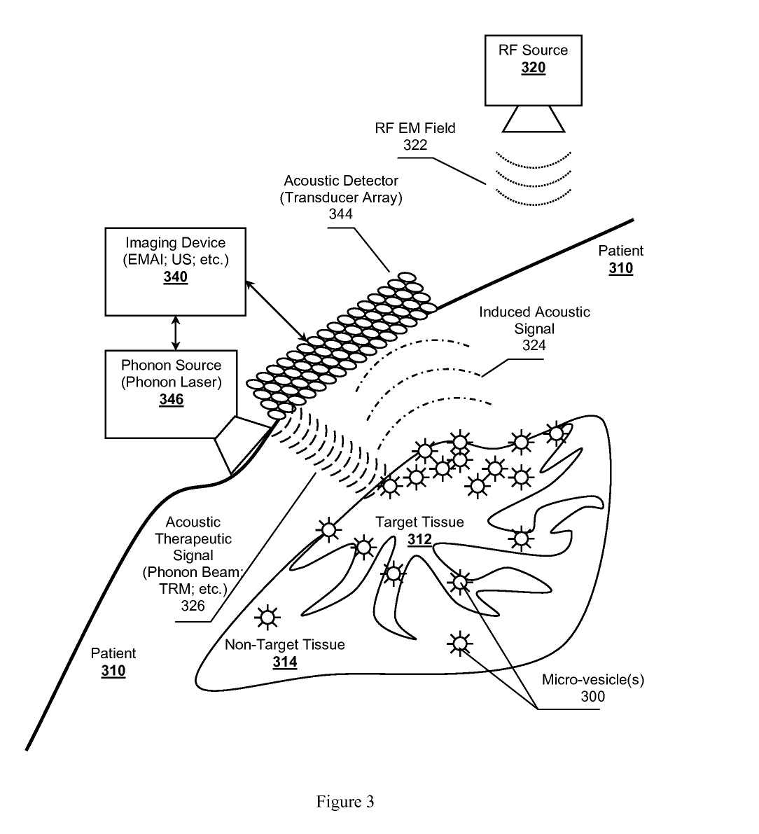

FIG. 3 presents an overview of how micro-vesicles 300 are used within an EMAI environment. Patient 310 has one or more internal tissues that require treatment or diagnosis as represented by target tissue 312. Micro-vesicles 300 have been constructed to preferentially attach to target tissue 312 over non-target tissue 314. One should note that micro-vesicles 300 can carry a drug formulation if desired.

In the EMAI environment, RF source 320 is configured to emit RF EM field 322. RF EM field 322 bathes target tissue 312 in RF energy. As discussed previously, RF EM field 322 can comprise a frequency across a very large range. Furthermore RM EM field 322 can include one or more peak frequency distributions. Target tissue 312, or other non-target tissue 314, generate an induced acoustic signal 324 in response to the RF EM field 322. Typically, induced acoustic signal 324 comprises a component having twice the frequency of RM EM field 322. One should appreciate that other components within induced acoustic signal 324 can also exist (e.g., harmonics, sum of input frequency peaks, difference between input frequency peaks, etc.). For example, when RF EM field 322 comprises a dominate input frequency of 5 MHz, induced acoustic signal 324 will included a ultrasound signal having a strong or dominate component at 10 MHz.

Induced acoustic signal 324 is representative of gradients in electrical properties of tissues 312 or 314. Micro-vesicles 300 can, at least in part, contribute to induced acoustic signal 324 as they can enhance contrast of an electrical property gradient (e.g., permittivity, conductivity, permeability, etc.).

Induced acoustic signal 324 can be collected via acoustic detector 324, which can comprise a transducer array. Collected acoustic data from acoustic detector 324 can be sent to imaging device 340, which analyzes the acoustic data and can present the acoustic data as image data on a display screen. Traditional approaches for displaying an ultrasound image can be used to derive an image from induced acoustic signal 324. In a preferred embodiment, imaging device 340 is a component of an EMAI device.

Providing contrast to target tissue 312 is only one use of micro-vesicles 300. In an EMAI environment, EMAI imaging device 340 can be used to locate a treatment site associated with target tissue 312 based on imaging data generated from induced acoustic signal 324. Once a treatment site is located, imaging device 340 can cause an acoustic therapeutic signal 326 to be generated and directed toward the treatment site.

Acoustic therapeutic signal 326 can be generated in numerous ways. In one embodiment, acoustic therapeutic signal 326 is a time reversed mirror (TRM) signal of the induced acoustic signal 324. A TRM signal essentially is a mirror of a received signal and is sent back to the point of origin. Therefore, when acoustic therapeutic signal 326 is a TRM signal, acoustic therapeutic signal 326 can be used to treat target tissue 312 with a high degree of precision by causing therapeutic excitation of micro-vesicles 300. The Applicant's own work describes using TRM acoustic signals in U.S. patent application having Ser. No. 12/786,232 titled “Time-Reversed Mirroring Electro-Magnetic Acoustic Treatment System” filed May 10, 2010, and in U.S. patent application having Ser. No. 12/913,586 titled “Dual Mode Imaging” filed Oct. 27, 2010. When acoustic therapeutic signal 326 comprises a TRM version of induced acoustic signal 324, the TRM signal can be amplified. The amplified TRM signal can be used to acoustically treat target tissue 312, by causing therapeutic excitation of micro-vesicles 300. Naturally, micro-vesicles 300 would enhance such treatment. Amplifying a TRM signal is described in the applicant's previously filing of U.S. patent application publication 2007/0038060 titled “Identifying and Treating Bodily Tissues Using Electromagnetically Induced, Time-Reversed, Acoustic Signals” filed Jun. 9, 2006.

In other embodiments, acoustic therapeutic signal 326 can be generated via phonon source 346, possibly operating as a phonon laser. An example phonon laser is described in U.S. Pat. No. 7,411,445 to Kucherov et al. titled “Phonon Laser”, filed Apr. 27, 2006, which describes a phonon laser pumped by a thermal gradient. Additional details of a phonon laser are described the paper by J. T. Mendonca, et al. titled “A phonon laser in ultra-cold matter” published in Europhysics Letters, 91 (2010) 33001. Acoustic oscillations from the phone laser can be coupled to the outside world via one or more transducer to create a highly directional targeted phonon beam. For example, phonon source 346 can couple with acoustic detector 344 (e.g., a transducer array), to create a phonon laser beam directed toward target tissue 312.

Regardless how acoustic therapeutic signal 326 is generated, acoustic therapeutic signal 326 can be directed toward target tissue 312 with a high degree of precision. Resolution of acoustic therapeutic signal 326 can have a resolution of better than about 5 mm axially (i.e., along the direction of propagation) and 2 mm laterally (i.e., orthogonal to the direction of propagation) based on the above techniques. Such precision provides for scanning a volumetric space associated with target tissue 312 and causing therapeutic excitation of only the micro-vesicles 300 within the beam. Thus, the only micro-vesicles 300 that become activated are those attached to a portion of target tissue 312 and in the presence of acoustic therapeutic signal 326.

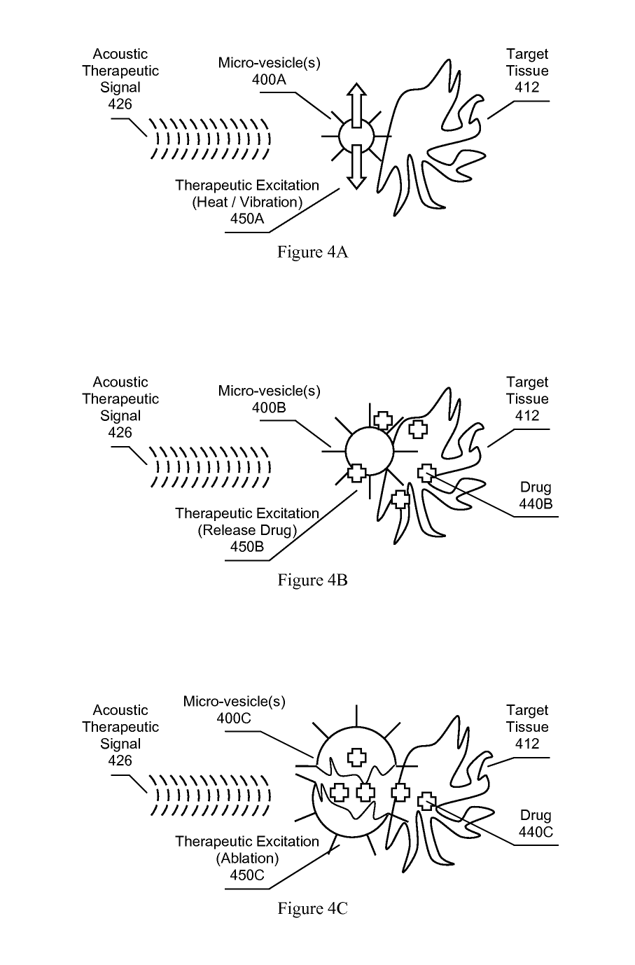

FIGS. 4A, 4B, and 4C illustrate various forms of therapeutic excitation of micro-vesicles in response to acoustic therapeutic signal 426. The therapeutic excitation provides treatment of target tissue 412 at a treatment site. In FIG. 4A, micro-vesicle 400A has been configured to be acoustically sensitive to acoustic therapeutic signal 426. In this example, micro-vesicle 400A has been configured to bind to target tissue 412 and to provide enhanced contrast to gradients associated with target tissue 412. Contrast is enhanced due to discontinuities of in a stress tensor related to materials composing the main body of micro-vesicle 400A. As a RF EM field impinges on target tissue 412, target tissue 412 and, to at least some degree, micro-vesicle 400A generates an induced acoustic signal.

In the example shown by FIG. 4A, micro-vesicle 400A can also become receptive to acoustic energy as shown. Acoustic therapeutic signal 426, possibly comprising an amplified TRM version of an induced acoustic signal, impinges micro-vesicle 400A. Micro-vesicle 400A can become resonantly activated as indicated by therapeutic excitation 450A, which can include heat or vibration. The heat from micro-vesicle 400A can be dissipated to target tissue 412, thus causing destruction of portions of target tissue 412.

One should note there are two triggering conditions to cause therapeutic excitation 450A, or other excitations (e.g., therapeutic excitation 450B, 450C, etc.). First, micro-vesicle 400A becomes activated in the presence of an RF EM field, which essentially pre-activates micro-vesicle 400A. Second, micro-vesicle 400A becomes receptive to acoustic therapeutic signal 426, which causes therapeutic excitation 450A. Thus, the disclosed approach provides for highly precise targeting of a treatment site or only small portions of a target tissue.

In FIG. 4B, micro-vesicle 400B has been configured to carry one or more drug formulations as represented by drug 440B on the surface of its main body. In this example, therapeutic excitation 450B is stemming from acoustic therapeutic signal 426 includes release of drug 440B. As acoustic therapeutic signal 426 impacts micro-vesicle 400B, drug 440B can be released via ablation of micro-vesicle 400B. One should note a surface material of micro-vesicle 400B does not necessarily have to ablate to release drug 440B. Rather, only drug 440B binding can be ablated and thus the drug released from the surface of micro-vesicle 400B.

In FIG. 4C, micro-vesicle 400C has been configured to carry one or more drug formulations as represented by drug 440C, which is held internally by micro-vesicle 400C. As acoustic therapeutic signal 426 impinges micro-vesicle 400C, assuming acoustic therapeutic signal 426 has sufficient amplitude or frequency, micro-vesicle 400C can be ruptured or broken to allow release of drug 440C.

After treating target tissue 412, micro-vesicles can be processed through the body through normal mechanisms. It is also contemplated that micro-vesicles can be flushed from the body through a normal filtering process. In some embodiments, for example where micro-vesicles comprise biocompatible materials, the micro-vesicle fragments can be dissolved or simply eliminated from the body. For example, porous silicon is a bio-degradable material, which can be naturally eliminated from the body. It should be appreciated that the particular fate of the micro-vesicles after treatment will predominantly be determined by the chemical composition of the micro-vesicles employed herein. For example, where the treatment includes disruption of the micro-vesicles, and where the fragments are not biodegradable, micro-vesicle fragments will typically be removed from the body via the reticuloendothelial system. On the other hand, where the fragments are biodegradable, micro-vesicle fragments will typically be degraded by various hydrolases and/or lipases, and further metabolized. In yet other aspects, where the micro-vesicles are predominantly formed from lipid layers (e.g., to form liposomes), filtration or resorption onto other membranes is contemplated.

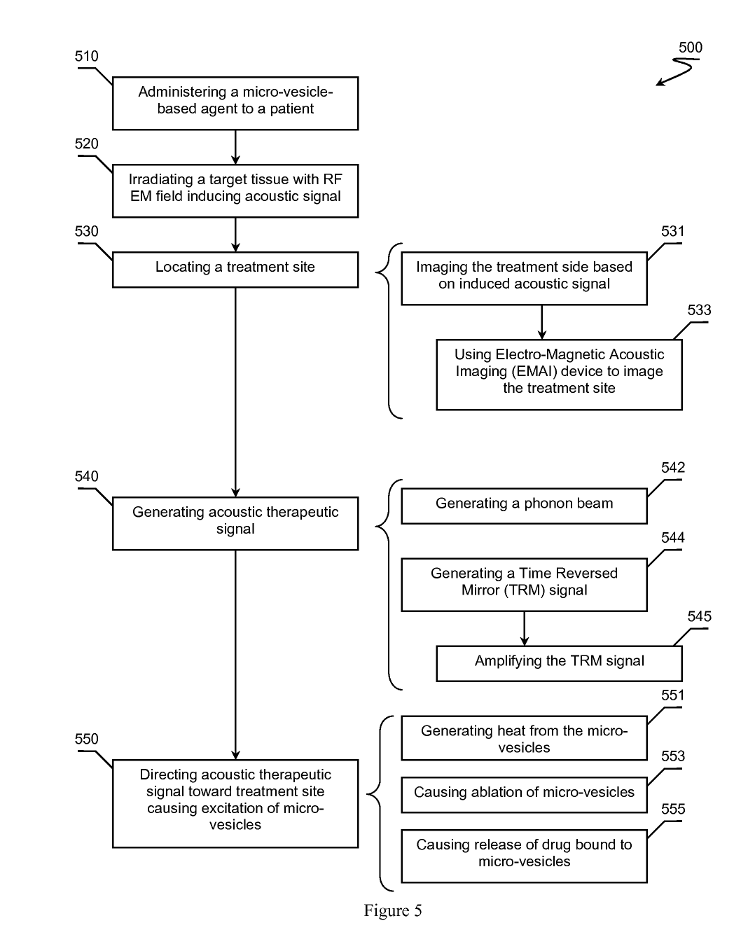

FIG. 5 presents a method 500 representing a possible process for treating a patient using a micro-vesicle-based agent. A micro-vesicle-based agent can include a composition having micro-vesicles as described previously. Step 510 can include administering the micro-vesicle-based agent to a patient. Administering can include injecting the agent, implanting, or otherwise introducing the agent to the patient. In some embodiments, the agent can be placed topically as opposed to being administered internally to the patient. When placed topically, the treatment environment can be adjusted by placing one or more additional acoustically conductive layers over the applied agent as needed before continuing.

Once a suitable amount of time has passed to allow micro-vesicles in the agent to bind to the target tissue, the process can continue. A suitable amount of time could range over a full spectrum of time from proceeding immediately to waiting hours, perhaps days. Step 520 includes irradiating the target tissue with an RF EM field, which in turn causes an induced acoustic signal to be generated due to electrical property gradients present in or near the target tissue. RM EM fields can be generated via one or more conducting coils (e.g., Helmholtz coils, MRI, etc.) operating at a desired frequency. Typically frequencies are in the range of 1 MHz to 20 MHz. However, higher frequencies are also contemplated, even up to 100 MHz, 500 MHz, 1 GHz, or even higher.

The induced acoustic signal typically has a frequency of twice the frequency of the RF EM frequency when a single dominant frequency peak is present and is embodied by an ultrasound signal. In more preferred embodiments, the induced acoustic signal is at least in part produced by micro-vesicles within the agent. The induced acoustic signal can be detected by an acoustic detector. Step 530 includes locating a treatment site associated with the target tissue by detecting the induced acoustic signal with the acoustic detector. For example, when a target tissue has a conductivity gradient, or other electrical property gradient and possibly including an enhanced contrast due to the presence of the micro-vesicles, the induced acoustic signal can be captured by an ultrasound transducer array. An imaging device can then provide information about the treatment site. In fact, step 531 can include imaging the treatment site at least partially based on the induced acoustic signal. Even further, step 533 can include using an EMAI device operating as the acoustic detection to conduct the step of imaging the treatment site. One should appreciate that locating the treatment site is not required to include imaging the treatment site or the target tissue. Furthermore, the inventive subject matter is considered to include providing enhanced electrical property contrast via administering micro-vesicles, especially where the micro-vesicles comprise a non-conducting main body outside the presence of an RF EM field.

When the treatment site is located, the process continues. Step 540 includes generating an acoustic therapeutic signal, which can include generating a phonon beam from a phonon laser as indicated by step 542. Furthermore, the acoustic therapeutic signal can also be generated by creating a TRM signal from the induced acoustic signal as suggested by step 544. Step 545 can include amplifying the TRM signal so that it has sufficient amplitude, or other properties, to interact with micro-vesicles at the treatment site.

Step 550 includes directing the acoustic therapeutic signal toward the treatment site, where the acoustic therapeutic signal causes a therapeutic excitation of the micro-vesicles bound to the target tissue. Directing the acoustic therapeutic signal can be achieved via one or more different methods. In some embodiments, the acoustic therapeutic signal can be constructed via a transducer array. Such a signal can be configured to have desirable properties targeting the treating sight including frequency, amplitude, phase, constructive interference, destructive interference, or other acoustic signal properties. An ultrasound transducer array can be used to generate a TRM version of an inbound signal where the TRM signal, by its nature, is directed back toward the point of origin. Naturally, the TRM signal can be tuned as desired via controlling aspects of the transducer array which in turn shapes the TRM signal. The TRM signal's properties can be adjusted as desired including the signal's amplitude, frequency or frequencies, phase, or other properties. One should appreciate that the array can shape the TRM signal, or other acoustic therapeutic signal, by controlling constructive or destructive interference of acoustic signals generated by elements of the array. Directing the acoustic therapeutic signal can also be accomplished by simply directing a phonon beam manually or automatically via one or more controllers. One should further appreciate that more than one acoustic therapeutic signal can be generated and directed toward to the treatment site via one or more acoustic therapeutic signal generators (e.g., multiple transducer arrays, phonon lasers, etc.).

Therapeutic excitation of micro-vesicles can include generating heat as referenced in step 551. As the acoustic therapeutic signal impinges the micro-vesicles, the micro-vesicles generate heat, which can be imparted to the target tissue to which the micro-vesicles are bound. Step 553 indicates that the therapeutic excitation can include causing ablation of micro-vesicles. In some embodiments, ablation of the micro-vesicles provides sufficient energy or treatment to a target tissue with or without a bound drug formulation. In other embodiments, as suggested by step 555, where the micro-vesicles include a bound drug formulation, the therapeutic excitation can include causing the release of the bound drug, possibly through ablation, through rupturing the micro-vesicle, or through fragmenting the micro-vesicle. One should also appreciate that the acoustic therapeutic signal can also directly heat or ablate the target tissue. Thus, the micro-vesicle can enhance such effects.

A number of experiments have been conducted to demonstrate that EMAI does indeed generate ultrasound with acceptable signal/noise. As two examples, EMAI generation was demonstrated from a conducting ball immersed in a saline solution thus providing a conductivity gradient, and from an insulator ball immersed in a saline solution thus providing a permittivity gradient. The conducting ball was roughly 1 cm in diameter and was made of RTV containing randomly ordered nickel wires. The insulator ball was also approximately 1 cm in diameter and was made of plastic. Both were immersed in a saline solution with an electrical conductivity of 2.5 S/m.

A test tube containing the saline solution with an immersed ball was placed in the mid-plane between two Helmholtz coils with coil diameters of 3 inches and a gap of 1.5 inches. The Helmholtz coils were excited at 5 MHz and had 1100 ampere turns. The ultrasound generated by the RF EM fields was detected by a lensed piezoelectric detector at a distance of 2 inches from the ball.

The measured ultrasound signals for the two balls at 10 MHz were 1.2 microvolts and 0.7 microvolts for the conducting RTV and plastic, respectively. These compared favorably with the predicted value (which did not depend on the diameters of the balls) and were of O(1 microvolt). Therefore, micro-vesicles configured with suitable electrical property values will also allow generation of an acoustic signal of the micro-vesicle in response to an external RF EM field. By suitability tuning the properties of the micro-vesicle, a generate acoustic signal can be at least a two-fold increase in strength relative to an acoustic signal of the target tissue induced in response to the external RF EM field.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A micro-vesicle comprising:
a main body comprising a core material and an affinity ligand coupled to the core material, wherein the affinity ligand has affinity to a target tissue; and
wherein the core material has an electrical property value sufficient to allow generation of an acoustic signal of the micro-vesicle in response to an external RF EM field that is at least a two-fold increase in strength relative to an acoustic signal of the target tissue in response to the external RF EM field.

2. The micro-vesicle of claim 1, wherein the main body is solid.

3. The micro-vesicle of claim 1, wherein the main body comprises an acoustically sensitive drug attachment site.

4. The micro-vesicle of claim 3, wherein the drug attachment site is configured to release a bound drug upon activation by an acoustic therapeutic signal.

5. The micro-vesicle of claim 4, wherein the drug attachment site is internal to the main body and the cavity is configured to release an enclosed drug upon exposing the main body to the acoustic therapeutic signal.

6. The micro-vesicle of claim 4, wherein the main body is configured to release the bound drug through ablation in response to the acoustic therapeutic signal comprising an ablative ultrasound signal.

7. The micro-vesicle of claim 4, wherein the main body is configured to release the bound drug in response to an amplified TRM signal of an internally sourced ultrasound signal at least partially generated by the main body in response to the applied RF electromagnetic field.

8. The micro-vesicle of claim 4, wherein the main body is configured to release the bound drug in response to a phonon laser beam.

9. The micro-vesicle of claim 1, wherein the affinity ligand comprises an antibody targeting the target tissue.

10. The micro-vesicle of claim 9, wherein the affinity ligand comprises a bi-specific antibody targeting the target tissue.

11. The micro-vesicle of claim 1, wherein the affinity ligand comprises at least one of a glycoprotein, a receptor specific ligand analog, and an antibody.

12. The micro-vesicle of claim 1, wherein the affinity ligand is configured with a preference for binding to the target tissue of at least 10 over a non-target tissue.

13. The micro-vesicle of claim 1, wherein the affinity ligand comprises an affinity for the target tissue of at least 10⁻⁴ per Mole.

14. The micro-vesicle of claim 1, wherein the material of the core material comprises an insulator when substantially free from exposure to the RF electromagnetic fields.

15. The micro-vesicle of claim 14, wherein the insulator material comprises one of the following materials: a glass, a ceramic, a porous silicon, a gel, and a polymer.

16. The micro-vesicle of claim 1, wherein the main body comprises a diameter of less than 100 nm.

17. The micro-vesicle of claim 1, wherein the electrical property is relative permittivity.

18. The micro-vesicle of claim 1, wherein the electrical property is magnetic permeability.

19. The micro-vesicle of claim 1, wherein the electrical property is electrical conductivity.

20. An injectable formulation comprising a quantity of micro-vesicles according to claim 1, wherein the micro-vesicles are present in the formulation in an amount that is effective to allow generation of an acoustically detectable signal at a plurality of cells or tissue to which the micro-vesicles are bound via the affinity ligand.

21. The injectable formulation of claim 20 wherein the main body of the micro-vesicles comprises an acoustically sensitive drug attachment site that is configured to release a bound drug upon activation by an acoustic therapeutic signal.
